(19) United States
(12) Patent Application Publication
(10) Pub. No.: US 2005/0143396 A1
(43) Pub. Date: Jun. 30, 2005

(54) Novel crystalline forms of ziprasidone hydrochloride

(75) Inventors: Reddy bandi Parthasaradhi, Hyderabad (IN); Reddy kura Rathnakar, Hyderabad (IN); Reddy rapolu Raji, Hyderabad (IN); Reddy dasari Muralidhara, Hyderabad (IN); Reddy itiyala Srinivas, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: HERTERO DRUGS LIMITED, ANDHRAPRADESH, IN (US)

(21) Appl. No.: 10/510,864

(22) PCT Filed: Apr. 11, 2003
(86) PCT No.: PCT/IN03/00154

(51) Int. Cl. A61K 31/519; C07D 487/04

(52) U.S. Cl. 514/259.41; 544/279

(57) ABSTRACT

The present invention provides novel crystalline forms of ziprasidone hydrochloride monohydrate, processes for their preparation and pharmaceutical compositions containing them.

### FIELD OF THE INVENTION

The present invention provides novel crystalline forms of ziprasidone hydrochloride monohydrate, processes for their preparation and pharmaceutical compositions containing them.

### BACKGROUND OF THE INVENTION

Ziprasidone of formula (1): 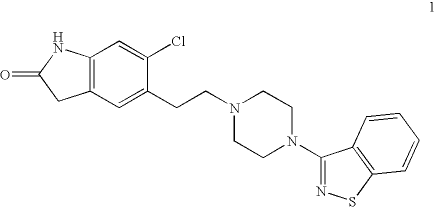
or 5-[2-[4-(1,2-Benzisothiazol-3-yl)-1-piperazinyl]ethyl]-6-chloro-1,3-dihydro-2H-indol-2-one and its salts are antipsychotic agents. Ziprasidone hydrochloride and related compounds and their therapeutic uses are disclosed in U.S. Pat. No. 4,831,031.

The crystalline forms of ziprasidone mesylate were reported in WO 97/42190, WO 97/42191.

It has now been discovered that ziprasidone hydrochloride monohydrate can be prepared in three stable crystalline forms having good dissolution characteristics.

The object of the present invention is to provide stable novel crystalline forms of ziprasidone hydrochloride monohydrate, processes for preparing these forms and pharmaceutical compositions containing them.

### DETAILED DESCRIPTION OF THE INVENTION

In accordance with the present invention, there is provided a novel crystalline form of ziprasidone hydrochloride monohydrate, designated as form I, characterized by an x-ray powder diffraction spectrum having peaks expressed as 2θ at about 10.9, 13.9, 15.9, 16.4, 17.5, 19.2, 20.6, 21.3, 21.9, 24.2, 24.7, 24.9, 25.7, 25.9 and 28.9 degrees. FIG. 1 shows typical form I x-ray powder diffraction spectrum.

In accordance with the present invention, a process is provided for preparation of ziprasidone hydrochloride monohydrate form I. Thus, a mixture of ziprasidone free base, hydrochloric acid and water is heated to about 45° C. to 100° C.; and ziprasidone hydrochloride monohydrate form I is isolated by filtration or centrifugation. Preferably, the mixture of ziprasidone free base, hydrochloric acid and water is heated to about 55° C. to 65° C.; and ziprasidone hydrochloride monohydrate form I is isolated by filtration or centrifugation.

In accordance with the present invention, there is provided a novel crystalline form of ziprasidone hydrochloride monohydrate, designated as form II, characterized by an x-ray powder diffraction spectrum having peaks expressed as 2θ at about 10.9, 11.3, 18.1, 19.5, 21.9, 23.7, 24.4, 24.8 and 26.2 degrees. FIG. 2 shows typical form II x-ray powder diffraction spectrum.

In accordance with the present invention, a process is provided for preparation of ziprasidone hydrochloride monohydrate form II. Thus, ziprasidone free base, an alcohol or a mixture of alcohols, dimethylformamide, a chlorinated solvent, hydrochloric acid and water are mixed to form a solution of ziprasidone hydrochloride; and the solvents are removed by the techniques such as vacuum drying, spray drying, freeze drying and lyophilization to form ziprasidone hydrochloride monohydrate form II. Water may be directly mixed or it may be mixed, for example, as an aqueous solution of hydrochloric acid. The alcohols are selected from the group consisting of methanol, ethanol, isopropyl alcohol, tert-butyl alcohol and n-butyl alcohol. The preferable alcohols are methanol and ethanol. The chlorinated solvents are selected from the group consisting of methylene dichloride, chloroform, carbon tetrachloride and ethylene dichloride. The preferable ester solvents are chloroform and methylene dichloride.

In accordance with the present invention, there is provided a novel crystalline form of ziprasidone hydrochloride monohydrate, designated as form III, characterized by an x-ray powder diffraction spectrum having peaks expressed as 2θ at about 10.9, 14.8, 15.9, 18.1, 19.5, 21.8, 24.3, 24.9, 25.9 and 26.5 degrees. FIG. 3 shows typical form III x-ray powder diffraction spectrum.

In accordance with the present invention, a process is provided for preparation of ziprasidone hydrochloride monohydrate form III. Thus ziprasidone free base, an ether solvent or a mixture of ether solvents, dimethylformamide, hydrochloric acid and water are mixed to form a solution of ziprasidone hydrochloride; and ziprasidone hydrochloride monohydrate form III is isolated from the solution. Water may be directly mixed or it may be mixed, for example, as an aqueous solution of hydrochloric acid. The ether solvents are selected from the group consisting of diethyl ether, diisopropyl ether and tert-butyl methyl ether. The preferable ether solvent is diethyl ether.

Ziprasidone free base used in the above processes can be obtained from the previously known methods.

In accordance with the present invention, there is provided a pharmaceutical composition comprising a crystalline form of ziprasidone hydrochloride monohydrate and pharmaceutically acceptable carrier or diluent. The crystalline form includes form I, form II or form III.

### BRIEF DESCRIPTION OF THE DRAWINGS

FIG. 1 is a x-ray powder diffraction spectrum of ziprasidone hydrochloride monohydrate form I.

FIG. 2 is a x-ray powder diffraction spectrum of ziprasidone hydrochloride monohydrate form II.

FIG. 3 is a x-ray powder diffraction spectrum of ziprasidone hydrochloride monohydrate form III.

x-Ray powder diffraction spectrum was measured on a Siemens D5000 x-ray powder diffractometer having a copper-Kx radiation.

The following examples further illustrate the present invention.

### EXAMPLE 1

Ziprasidone free base (10 gm), conc. hydrochloric acid (10 ml) and water (150 ml) are mixed and the reaction mass is heated to 60° C. and stirred for 4 hours at 60° C. to 65° C. The contents are cooled to 25° C., filtered, washed with water and dried to give 10 gm of ziprasidone hydrochloride monohydrate form I.

### EXAMPLE 2

Ziprasidone freebase (2.5 gm), methanol (100 ml), dimethylformamide (100 ml), chloroform (25 ml) and conc. hydrochloric acid (1.5 ml) are mixed at 25° C. The contents are heated to 60° C. and stirred for 10 minutes at 60° C. to 65° C. and the clear solution thus obtained is subjected to vacuum drying at 70° C. for 40 hours to give ziprasidone hydrochloride monohydrate form II in near quantitative yield.

### EXAMPLE 3

Ziprasidone free base (3.0 gm), methanol (120 ml), dimethylformamide (100 ml), chloroform (30 ml) and conc. hydrochloric acid (1.5 ml) are mixed at 25° C. The contents are heated to 60° C. and stirred for 10 minutes at 60° C. to 65° C. and the clear solution thus obtained is subjected to spray drying to give ziprasidone hydrochloride monohydrate form II.

### EXAMPLE 4

Ziprasidone free base(5.0 gm) is added to diethyl ether (50 ml) and heated to reflux temperature. Then dimethylformamide (145 ml) is added and the contents are stirred for 2 hours under reflux. Then conc. hydrochloric acid (2.5 ml) and water (3 ml) are added, and the solution is cooled to 25° C. The separated crystals are filtered to give 3.5 gm of ziprasidone hydrochloride monohydrate form III.

1. A crystalline ziprasidone hydrochloride monohydrate form I, characterized by an x-ray powder diffraction spectrum having peaks expressed as 2θ at about 10.9, 13.9, 15.9, 16.4, 17.5, 19.2, 20.6, 21.3, 21.9, 24.2, 24.7, 24.9, 25.7, 25.9 and 28.9 degrees.

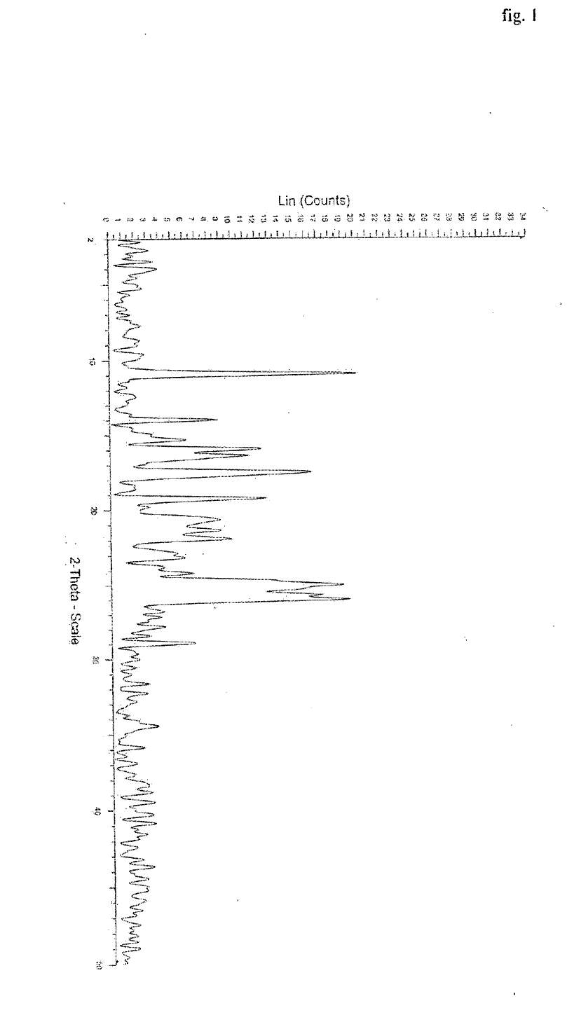

2. A crystalline ziprasidone hydrochloride monohydrate form I, as defined in claim 1, further characterized by an x-ray powder diffraction spectrum as in FIG. 1.

3. A process for preparation of ziprasidone hydrochloride monohydrate form I as defined in claim 1, which comprises the steps of:
a) mixing ziprasidone free base, hydrochloric acid and water;
b) heating to about 45° C. to 100° C.; and
c) isolating ziprasidone hydrochloride monohydrate form I by filtration or centrifugation.

4. The process according to claim 3, wherein the reaction mass is heated to about 60° C. to 65° C. in (b).

5. A crystalline ziprasidone hydrochloride monohydrate form II, characterized by an x-ray powder diffraction spectrum having peaks expressed as 2θ at about 10.9, 11.3, 18.1, 19.5, 21.9, 23.7, 24.4, 24.8 and 26.2 degrees.

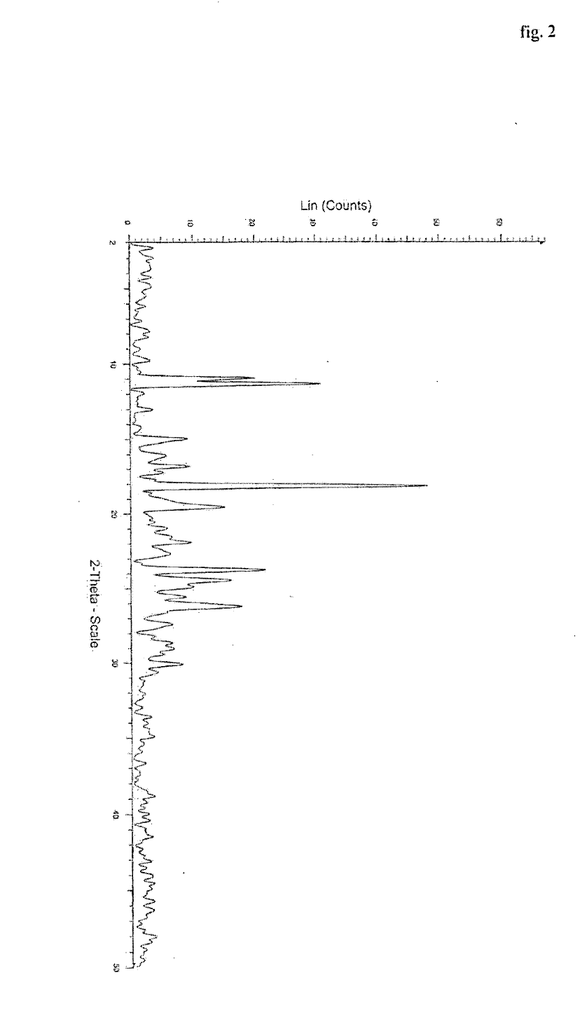

6. The crystalline ziprasidone hydrochloride monohydrate form II as defined in claim 5, further characterized by an x-ray powder diffraction spectrum as in FIG. 2.

7. The process for preparation of ziprasidone hydrochloride monohydrate form II as defined in claim 5, which comprises the steps of:
a) mixing ziprasidone free base, an alcohol or a mixture of alcohols, dimethylformamide, a chlorinated solvent, hydrochloric acid and water to form a ziprasidone hydrochloride solution; and
b) removing the solvents from the solution;
wherein the alcohol is selected from the group consisting of methanol, ethanol, isopropyl alcohol, tert-butyl alcohol and n-butyl alcohol; and the chlorinated solvent is selected from the group consisting of methylene dichloride, chloroform, carbon tetrachloride and ethylene dichloride.

8. The process according to claim 7, wherein the solvents are removed by the techniques such as vacuum drying, spray drying, freeze drying and lyophilization.

9. The process according to claim 7, wherein the alcohol is methanol.

10. The process according to claim 7, wherein the chlorinated solvent is chloroform.

11. A crystalline ziprasidone hydrochloride monohydrate form III, characterized by an x-ray powder diffraction spectrum having peaks expressed as 2θ at about 10.9, 14.8, 15.9, 18.1, 19.5, 21.8, 24.3, 24.9, 25.9 and 26.5 degrees.

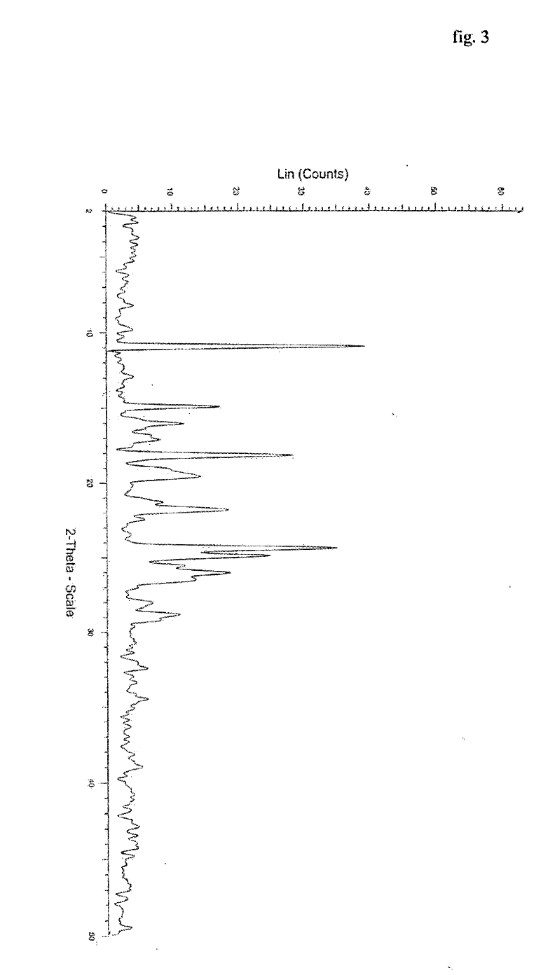

12. The crystalline ziprasidone hydrochloride monohydrate form III as defined in claim 11, further characterized by an x-ray powder diffraction spectrum as in FIG. 3.

13. The process for preparation of ziprasidone hydrochloride monohydrate form III as defined in claim 11, which comprises the steps of:
a) mixing ziprasidone free base, an ether solvent or a mixture of ether solvents, dimethylformamide, hydrochloric acid and water to form a ziprasidone hydrochloride solution; and
b) isolating ziprasidone hydrochloride monohydrate form III from the solution; wherein the ether solvent is selected from the group consisting of diethyl ether, diisopropyl ether and tert-butyl methyl ether.

14. The process according to claim 11, wherein the ether solvent is diethyl ether.

15. The pharmaceutical composition comprising ziprasidone hydrochloride monohydrate form I of claim 1 and a pharmaceutically acceptable carrier or diluent.

16. The pharmaceutical composition comprising ziprasidone hydrochloride monohydrate form II of claim 5 and a pharmaceutically acceptable carrier or diluent.

17. The pharmaceutical composition comprising ziprasidone hydrochloride monohydrate form III of claim 11 and a pharmaceutically acceptable carrier or diluent.
